(19) United States
(12) Patent Application Publication
(10) Pub. No.: US 2026/0275262 A1
(43) Pub. Date: Sep. 17, 2026

(54) CELL SUBSTRATE FOR RECEIVING BIOLOGICAL CELLS, AND METHOD FOR USE THEREOF

(71) Applicant: CHANPHARM GMBH, Vienna (AT)

(72) Inventor: Steffen HERING, Vienna (AT)

(21) Appl. No.: 19/508,507

(22) PCT Filed: Jun. 26, 2024
(86) PCT No.: PCT/EP2024/067978

§ 371 Date: Feb. 4, 2026

(30) Foreign Application Priority Data
Jul. 3, 2023 (DE) 10 2023 117 490.8

(51) Int. Cl. C12M 1/12; C12M 1/42

(52) U.S. Cl. CPC C12M 25/14; C12M 35/02

(57) ABSTRACT

A cell substrate 100 configured to receive biological cells 1 includes a substrate body 10 having a structured substrate surface 20, wherein the structured substrate surface 20 has a plurality of cell-receiving segments 21 arranged side-by-side, each having a longitudinal extension, adjacent cell-receiving segments 21 are each separated by a wall region 22 projecting from the substrate body 10, and the wall regions 22 between the adjacent cell-receiving segments 21 each have a wall profile bounded by segment wall surfaces perpendicular to the longitudinal extension of the adjacent cell-receiving segments 21, wherein the wall profile of the wall regions 22 between the adjacent cell-receiving segments 21 each has a wall tip 23 projecting from the substrate body 10, which suppresses a cell arrangement on the wall region 22. Uses of the cell substrate 100 are also described.

### FIELD OF THE INVENTION

The invention relates to a cell substrate which is configured to receive and preferably to examine biological cells, particularly preferably for electrophysiological (electrical) measurement on the biological cells. Furthermore, the invention relates to methods for using the cell substrate, e.g. in the arrangement, cultivation and/or examination of biological cells, in particular of heart muscle cells (cardiomyocytes) or nerve cells (neurons).

### TECHNICAL BACKGROUND

In the present description, reference is made to the following prior art, which illustrates the technical background of the invention:
[1] A. S. T. Smith et al. in “Nano Lett.” 2020, 20, 3, pp. 1561-1570;
[2] M. Kitsara et al. in “Microelectronic Engineering” 2019, 203-204, pp. 44-62;
[3] H. Savoji et al. in “Biomaterials” 2019, 198, pp. 3-26;
[4] V. J. Kujala et al. in “J. Mater. Chem.” B, 2016, 4, p. 3534;
[5] A. Atmanli et al. in “J. Vis. Exp.” (73), e50288, doi:10.3791/50288(2013 );
[6] WO 2021/083885A 1;
   [7] T. C. McDevitt et al. in “J. Biomed. Mater. Res.”, 2002, 60, 472-479;
   [8] G. Khademhosseini et al. in “Biomed. Microdevices”, 2007, 9, 149-157;
   [9] Ma et al. in “Lab Chip” 2012, 12, 566;
   [10] WO 2014/074067A 1;
   [11] F. Navaee et al. “Cells” 2023, 12, 576;
   [12] J. P. Kucera et al. in “Circ Arrhythm Electrophysiol.” 2017; 10:e 004665;
   [13] Bartels et al. in “Sci Rep.” 2021; 11:9269;
   [14] M. Hippler et al. in “Sci Adv.” 2020 Sep; 6(39): eabc 2648.
   [15] K. Weißenbruch et al. in “Curr Opin Biotechnol.” 2022 Feb; 73:290-299;
   [16] Q. Akolawala et al. in “ACS Appl. Mater. Interfaces” 2022, 14, 18, 20778-20789;
   [17] S. H. Yagoub et al. in “Journal of Assisted Reproduction and Genetics” vol. 39, pp. 1503-1513 (2022); and
   [18] DE 10 2008 047 399 A1.

It is generally known that the provision of differentiated cells, such as heart cells or neurons, through the differentiation of human pluripotent stem cells (hPSC) enables the development of tissue models, such as heart muscle or nerve tissue-like tissues. Tissue models can be used in drug development to investigate the effect of drugs on tissue (“tissue-on-a-chip” technologies). For example, electrophysiological measurements and/or optical measurements and/or measurements of heart cell contractions can be carried out on heart cells using high-throughput methods (see e.g. [1] to [3]).

There is an interest in examining the differentiated cells in a condition that is as similar as possible to the cell conditions in a natural tissue environment. For example, the development of “heart-on-a-chip” technologies may involve growing heart cells on anisotropic substrate structures to mimic a fiber structure in the myocardium (see e.g. [1], [2]). Testing of drugs on laminar, fibrous cardiac tissue structures on groove structures or on directed nerve fibers in grooves has made initial progress in the testing of drugs (see e.g. [2], [4] or [11]).

For example, receiving of the heart cells on a cell substrate with a plurality of surface grooves (hereinafter also referred to as cell-receiving segments) was realized, in which a fibrous arrangement and alignment of the heart cells is generated (see e.g. [1], [4] and [5]), whereby adjacent grooves are separated by intermediate walls. Surface grooves are formed, for example, using printing methods with a height in a range from 1 μm to 100 μm, a width in a range from 1 μm to 100 μm and a length of 1 mm or more. The intermediate walls consist, for example, of a polymer such as PDMS or a hydrogel.

Alternatively, grooves are created by etching recesses in a substrate surface, micro-injection molding or lithographic processes (see e.g. [1], [2]) or by micro-contact printing or by means of microfluidic structures. Furthermore, cell adhesion proteins (e.g. fibronectin, laminin or collagen) may be used for the alignment of cardiac cells for the formation of cardiac muscle fibers in vitro, which cell adhesion proteins are applied to e.g., polystyrene cell culture dishes, in order to predetermine the alignment of the cardiomyocytes ([7], [8]).

It is also known to use substrates with integrated electrodes for electrophysiological investigations. For example, a substrate is described in [6] in which tip-shaped nanoelectrodes are arranged at the bottom of cell-receiving segments, which penetrate into biological cells when they are received on the substrate. The nanoelectrodes can be disadvantageous if they require an additional setting device on the substrate to penetrate the cell membrane. Another disadvantage may be that the nanoelectrodes are designed for intracellular deriving of membrane potentials and not for the measurement of surface potentials.

However, there is great interest in the measurement of surface potentials, as the temporal length of field potentials and the shape of measured T-waves (such as determining the prolongation of the ECG parameter Q-T time) on fiber surfaces are highly informative with regard to drug effects and are similar to an ECG measurement. Measurements of surface potentials are possible by using planar electrodes arranged at the bottom of the surface grooves of a cell substrate.

Cell substrates with flat electrodes can be produced by forming an anisotropic substrate structure, for example with surface grooves, on a so-called micro electrode array (MEA) substrate (or MEA chip) (see e.g. [9]). The MEAs are exposed at the bottom of the surface grooves. By positioning several electrodes at the bottom of a groove (see e.g. [9]), the propagation of an excitation along a cell fiber, e.g. a myocardial or nerve fiber, can be determined. This makes the use of fluorescence probes molecules for measurements of action potentials advantageously obsolete. Since the use of fluorescence probes can lead to cell-toxic degradation products and these fluorescence probes can also influence the effects of drugs, the direct determination of excitation propagation is particularly useful for drug research.

However, one disadvantage of known MEAs is that heart muscle cells growing as monolayer (cell lawn), which are connected to each other via cell-cell contacts (nexus contacts), detach from the substrate after a relatively short time. Cell lawns are initially connected to the substrate via attachment points distributed over a large area. During the rhythmically occurring contractions, the cells stretch the cell lawn in different spatial directions. However, force effects in the different spatial directions quickly lead to detachment from the substrate. This leads to an unwanted shortening of the time periods available for investigations.

Another disadvantage of known cell substrates with a groove structure on a MEA substrate is the insufficient mutual partitioning (separation, isolation) of individual fibers in adjacent grooves. Single fibers in adjacent grooves can unintentionally touch each other at different (undefined) positions and spontaneously form cell-cell contacts when cells adhere to the top of the intermediate wall between the grooves and grow over into the adjacent groove. Cells are located, for example, as a result of the deposition of the cells 1′ on the cell substrate 100′ and/or by a cell growth or cell migration via the intermediate wall 22′, on the upper side of the intermediate wall (see FIG. 11, prior art). In known cell substrates, intermediate walls and associated upper sides of the intermediate walls of conventional cell substrates with grooves 21′ have wall thicknesses in the range from 10 μm to 30 μm to achieve sufficient stability, so that adhesion of cells with a diameter of 10 μm is promoted.

However, electrophysiological measurements can be distorted by unintentional, irregular contact of individual fibers from adjacent grooves. The cells on e.g. 5 μm to 100 μm high wall structures can establish electrical contacts between adjacent fibers. Excitations of individual fibers (e.g. spontaneously or by electrical stimulation) are then conducted spontaneously and undefined to adjacent fibers, so that a measurement of the excitation conduction along a defined section of a single fiber with the surface electrodes belonging to one groove is excluded or at least falsified.

Furthermore, the irregular overgrowth of wall structures over several grooves forms tissue segments which, due to their size, increase the undesirable force effects during contraction movements in different spatial directions, so that the unintentional detachment from the substrate is further accelerated.

Finally, the spontaneous and uncontrolled adhesion of cells to the wall surfaces and the transfer of excitation processes (“crosstalk”) to adjacent fibers also prevent the reliable subdivision of cell culture surfaces into separate, well-defined sections.

Studies in practice have shown that cell-repellent nanostructures on the intermediate walls or a coating with strong hydrophobicity can reduce cell growth on the upper side of the intermediate wall, but cannot prevent spontaneous overgrowth of the walls. For example, the structures described in [6] (see e.g. FIG. 12, cited from [6]) still provide sufficient surfaces for adherent attachment of cells and overgrowth of the intermediate walls 22′. The overgrowth of the upper side of the intermediate wall can be promoted, inter alia, by the fact that the cells deposited on the flat upper side synthesize extracellular matrix proteins, which then enable cell adhesion even on cell-repellent surfaces.

The above-mentioned disadvantages due to the adherent attachment of cells and the overgrowth of the intermediate walls do not only affect electrophysiological investigations. Other applications of the cell substrate, from the pure cultivation of the cells to other, e.g. optical or chemical, investigations can also be impaired by cells, e.g. on upper sides of the cell wall.

In the practical application of conventional cell substrates with surface grooves, it was found to be a further disadvantage that the filling of the grooves can be more difficult if cells of a suspension do not reach the bottom of grooves and associated electrodes, but adhere to the intermediate walls of the grooves.

### OBJECTIVE OF THE INVENTION

The objective of the invention is to provide an improved cell substrate to receive biological cells which is suitable for avoiding disadvantages of conventional techniques. In particular, the objective of the invention is to provide a cell substrate which separates cells in different cell-receiving segments with increased reliability and/or reproducibility, better suppresses or excludes an uncontrolled mutual interaction of cells in different cell-receiving segments, prolongs the duration and/or adhesion of the biological cells on the cell substrate, improves the reliability and informative value of investigations on the cells on the cell substrate, and/or enables new applications of MEA substrates. The objective of the invention is also to provide improved applications of the cell substrate to receive biological cells.

### SUMMARY OF THE INVENTION

These objectives are solved by a cell substrate to receive biological cells having the features of claim 1. Preferred embodiments and applications of the invention result from the dependent claims.

According to a first general aspect of the invention, the above objective is solved by a cell substrate configured to receive biological cells and comprising a substrate body having a structured substrate surface. The structured substrate surface has a plurality of cell-receiving segments arranged side-by-side, each having a longitudinal extension. Adjacent cell-receiving segments are each separated by a wall region extending from the substrate body and bounded by segment wall surfaces. The wall regions between the adjacent cell-receiving segments each have a wall profile (cross-sectional shape) perpendicular to the longitudinal extension of the adjacent cell-receiving segments.

According to the invention, the wall profile of the wall regions between the adjacent cell-receiving segments each has a wall tip projecting from the substrate body, which suppresses a cell arrangement on the wall regions. Along the length of the wall regions, the respective wall profile with the wall tip can preferably form one, particularly preferably a single wall vertex line (or: wall edge), which is shaped as a barrier against cell deposition on the wall region and/or against cell growth or a cell migration between adjacent cell-receiving segments. With the wall edge, a cell arrangement on the wall region can be suppressed. Wall profiles of the wall regions can be shaped in such a way that the wall regions adjacent to a cell-receiving segment form a funnel-shaped inlet into the cell-receiving segment (funnel slit). Advantageously, this can support a collection and/or concentration of cell material at the bottom of cell-receiving segments, in particular during centrifugation of a cell suspension on an MEA substrate with cell-receiving segments.

According to a second general aspect of the invention, the above objective is solved by a method for using the cell substrate according to the first general aspect of the invention or one of its embodiments, wherein at least one of the following steps is provided: cultivation of biological cells in the cell-receiving segments of the cell substrate, examination of the biological cells, in particular measurement of electrophysiological potentials on biological cells in the cell-receiving segments of the cell substrate, determination of an excitation propagation speed on cells, e.g. fibers or cell extensions (in particular axons and/or dendrites) along at least one of the cell-receiving segments of the cell substrate by a detection of a time-delayed arrival of an electrical excitation at electrodes at the bottom of the cell-receiving segment, in particular without a fluorescence measurement, and determination of an excitation propagation speed through cells along at least one of the cell-receiving segments of the cell substrate, in particular with simultaneous fluorescence measurement for forwarding an action potential with a potential-sensitive fluorescence probe and the intracellular calcium release with a calcium-sensitive fluorescence probe, cell contraction with video microscopy, optical measurement of a contraction of cardiac muscle cells by analyzing the shortening of cell segments, measurements of intracellular calcium concentrations, electrical stimulation of biological cells in the cell-receiving segments of the cell substrate, biochemical stimulation of biological cells in the cell-receiving segments of the cell substrate, and operation of the cell substrate as a generator device adapted to generate electrical current by membrane potential discharge at the biological cells in the cell-receiving segments of the cell substrate (see [18]). The electrophysiological determination of the excitation propagation speed can advantageously be used to determine a nexus resistance of cell-cell contacts, in particular without a fluorescence measurement.

The cell substrate is generally formed by the substrate body with the structured substrate surface. The substrate body can be plate-shaped, preferably plane and/or solid (rigid). The substrate body generally comprises an underside, preferably formed by a substrate plate, such as an MEA substrate and/or a substrate with integrated microfluidics for supplying cells on the cell substrate, and an upper side on which the structured substrate surface to receive the cells is exposed. The substrate body and the structured substrate surface can be formed in one piece or assembled (in particular in multiple layers) and/or made of plastic, a glass, a semiconductor and/or a ceramic. The substrate body and/or the structured substrate surface can be formed from a transparent material, which advantageously enables microscopic observation and/or examination of cells on the cell substrate to be simplified.

The cell-receiving segments are formed by the shape of the structured substrate surface. Each cell-receiving segment is generally a recess, in particular a depression in the substrate surface and/or a receptacle formed by protruding wall regions, which has the longitudinal extension. The longitudinal extension of a cell-receiving segment refers to a geometric shape with which the cell-receiving segment extends mainly in one direction (longitudinal direction of the cell-receiving segment, length), while the extension of the cell-receiving segment along the substrate surface perpendicular to the longitudinal direction (lateral extension of the cell-receiving segment, width) is smaller than the length. For example, the width of the cell-receiving segments can be equal to or less than ⅕, in particular 1/10 of the length of the cell-receiving segments.

Preferably, the cell-receiving segments can have a straight shape, either completely or in sections, i.e. at least one of the cell-receiving segments can form a straight, linear receptacle (also referred to as a groove, channel or slot) in part or particularly preferably along its entire length. Alternatively, at least one of the cell-receiving segments can be bent (curve-shaped) at least in sections. In the case of the continuously straight cell-receiving segment, the longitudinal extension can be formed by a straight groove shape, and in the case of a cell-receiving segment that is curved in sections or continuously, the longitudinal extension can be formed by a locally curved groove shape.

The areas of the structured substrate surface by which the directly adjacent cell-receiving segments are separated from each other are referred to as wall regions. In the case of a curved cell-receiving segment, wall regions are alternatively or additionally provided between various preferably straight segment sections of the cell-receiving segment.

A wall region is generally a protrusion projecting from the substrate body, which has a longitudinal extension and has mutually adjacent cell-receiving segments (or different adjacent segment sections of curved cell-receiving segments) on both sides and is bounded by the segment wall surfaces. A wall region may have a thickness (extension perpendicular to the longitudinal direction of the bounding cell-receiving segments) which may be less than, equal to or greater than the width of the adjacent wall regions and/or less than, equal to or greater than the depth of the bounding wall regions. The wall regions may be configured for an uninterrupted continuous partition of the adjacent cell-receiving segments along their longitudinal extension. The term “partition” of the adjacent cell-receiving segments optionally also includes a boundary formed in sections along the longitudinal extension of the cell-receiving segments, which may have through-openings (see below).

In general, each wall region between adjacent cell-receiving segments (or segment sections of bent cell-receiving segments) forms a wall (segment wall) between the cell-receiving segments (or their segment sections). The cross-sectional shape of a wall region transverse to the longitudinal direction of the bounding cell-receiving segments forms the wall profile of the wall region. Advantageously, at least one of the wall regions, preferably all wall regions, can have a constant wall profile along the respective longitudinal extension of the wall region over its entire length. Alternatively, the wall profile of a wall region and a surface structure of the wall can be variable along its longitudinal extension, for example forming a narrower or wider receptacle at the longitudinal ends of the bounding cell-receiving segments than in a central part of the cell-receiving segments. Alternatively or additionally, all wall profiles of all wall regions can have the same shape or different shapes.

The wall profile can be formed by sloping side surfaces (segment wall surfaces) on the sides of the wall region that are tapered or parallel to the wall tip. The wall profile can preferably have a continuous triangular shape with the wall tip along the entire length of the respective wall region. The triangular shape, including the protruding wall tip, refers to a shape of the wall profile that can be described by three points. The three points include a first point at an end of the wall region facing away from the cell substrate, where the wall tip is formed, and two (base) points on the lateral surfaces of the wall region in the depth of the bounding cell-receiving segments. The wall profile can have a straight contour shape or a curved contour shape between the aforementioned points, i.e. the lateral surfaces of the wall regions (segment walls) can form a straight or curved, in particular concave, line. Alternatively or additionally, the wall profile can be formed by a vertical ridge (also referred to as a partition or lamella) that protrudes from the substrate body. The inventor has found that the wall tips and/or vertical ridges can be produced with high stability, so that they can have a considerably smaller thickness than conventional intermediate walls, e.g., in the range 0.5 μm to 20 μm.

At the wall tip (optionally at the free end of a vertical ridge, see below), an extremely small radius of curvature equal to or smaller than 10 μm, in particular equal to or smaller than 5 μm, e.g. 1 μm or smaller, up to 100 nm, can be set, whereby a cell overgrowth between adjacent cell-receiving segments is excluded. Alternatively, this effect can also be achieved with a larger radius of curvature, e.g. in the range from 10 μm to 15 μm or from 10 μm to 20 μm or more.

Along the length of a respective wall region, the wall tip of the wall profile forms a continuous wall vertex line, at least in sections, which is preferably exposed and/or, as explained below, can be formed by a vertical ridge.

The inventors have found that the design of the wall regions with the wall profile, which forms the wall vertex line, advantageously creates a cell barrier between bounding cell-receiving segments. The surface of the wall region has a shape that hinders down to a negligible extent or completely prevents the deposition of cells when cells are deposited on the cell substrate and/or hinders down to a negligible extent or completely excludes cell growth or cell migration from one side of the wall region via the upper side of the wall region to the other side of the wall region. Cell deposition, cell growth and/or cell migration are hindered down to a negligible extent if cells or cell components remaining on the upper side of the wall regions do not influence the application of the cell substrate, in particular a cultivation of the cells and/or an examination of the cells in the cell-receiving segments.

A particular advantage of the invention is that the shape of the wall regions according to the invention creates a compartmentalization of the structured substrate surface of the cell substrate, i.e., each cell-receiving segment forms an elongated vessel (compartment) for the cells received, which is separate from other cell-receiving segments. Advantageously, the invention creates a subdivision of the structured substrate surface into individual elongated cell receptacles, which can be open at the upper side and are separated from one another without the individual cell receptacles having to be separated from one another by lids. A further advantage of compartmentalization is that the yield is improved when cells are used for cell deposition on the substrate, for example in that all cells received on the cell substrate contribute to fiber formation.

The above-described disadvantages of conventional cell substrates with surface grooves for receiving and cultivating biological cells, in particular heart and/or nerve cells, are remedied by the wall regions provided according to the invention. When depositing (in particular seeding) a cell suspension, cells can be prevented from adhering to the upper surface of the wall regions. At the same time, inclined side walls adjacent to a cell-receiving segment can form an elongate funnel slit, which facilitates the filling of the cell-receiving segments, since the cells in the suspension inevitably collect at the bottom of the cell-receiving segments, e.g. under the effect of gravity and/or a centrifugal force, and are guided towards the bottom by the segment wall surfaces of the wall regions. The shape of the structured substrate surface according to the invention thus not only provides a partition of the biological cells in the cell-receiving segments, but also a filling aid for the cell substrate. Biological cells can be arranged more uniformly and with increased reliability in the cell-receiving segments.

Advantageously, a micro-compartmentalization of the substrate surface can be provided according to the invention, which limits the amplitude of the contractions to one direction (the longitudinal direction of the cell-receiving segments). As a result, the risk of an early detachment of the cells due to contractions can be substantially reduced, as fewer lateral contractions and/or elongations occur. Due to the longitudinal adhesion in the cell-receiving segments, the duration of the adhesion of cells to the surface is extended, whereby lateral contacts on the walls of the cell-receiving segments have an additional stabilizing effect.

According to a preferred embodiment of the invention, the segment wall surfaces of at least one of the wall regions, preferably all of the wall regions, may enclose an acute angle distally at the wall tip between adjacent cell-receiving segments or may run parallel. The acute angle is an angle of less than 90°, preferably less than 80°. The smaller the angle at the wall tip, e.g. down to 0°, the more effective the partitioning of the adjacent cell-receiving segments is advantageously. Both segment wall surfaces of a wall region can be inclined relative to the substrate surface. In particular, if they enclose the acute angle, the segment wall surfaces can be inclined relative to each other and/or relative to the extension of the substrate body (inclined segment wall surfaces). Alternatively, one of the segment wall surfaces of a wall region may be inclined relative to the substrate surface, while the other segment wall surface of the wall region is perpendicular relative to the substrate surface. According to a further alternative, both segment wall surfaces of a wall region may be perpendicular relative to the substrate surface, i.e. the wall region can form the vertical ridge (see below).

According to a further preferred embodiment of the invention, the segment wall surfaces can have a low roughness such that structures (protrusions and/or depressions) on the segment wall surfaces have dimensions ≤100 nm, e.g. ≤50 nm, in particular ≤10 nm. Irregular, fissured surfaces on μm or nm scales and thus increased adhesion of cells can thus be advantageously suppressed or avoided.

Advantageously, the segment wall surfaces and/or the wall tip along the length of the wall regions can be free of local structures, such as local tips, which could form a rough topology in particular.

According to a further preferred embodiment of the invention, at least one, particularly preferably each, of the wall regions can have a vertical ridge (lamella) projecting from the substrate body, which forms the wall tip in the wall profile of the wall region. The shape of the wall profile can be selected such that the wall tip is formed by the vertical ridge (in particular a protruding lamella). Preferably, the vertical ridge can extend over the entire height of the wall region, i.e. form an ultra-thin wall with the parallel segment wall surfaces. Alternatively, the partition of adjacent cell-receiving segments (or segment section) can be formed by a vertical ridge arranged on a triangular profile base area of a wall region.

Preferably, the thickness of the vertical ridge can be selected in the range from 1 μm to 10 μm, for example 1 μm to 5 μm. Alternatively, the thickness of the vertical ridge can be less than 1 μm. The provision of the vertical ridge has the advantage that the partition of the adjacent cell-receiving segments or sections of a curved cell-receiving segment is improved. In particular, the vertical ridge provides an insurmountable barrier to cell growth or cell migration from the cell-receiving segments.

Additional advantages of the cell substrate may result if, according to a further embodiment of the invention, the segment wall surfaces adjacent to at least one of the cell-receiving segments comprise lateral protrusions facing each other and forming a segment slit extending along the longitudinal extension of the cell-receiving segment.

The lateral protrusions can advantageously be formed in such a way that the wall profile of the wall regions has an arrow shape, the distal end of which forms the wall tip, optionally with the vertical ridge. In this case, the cross-sectional shape of a wall region can be composed of a triangle with the wall tip and a rectangle in the lower part of the wall region.

Unlike in [6] (see FIG. 11), the lateral protrusions are not formed by T-profiles, but preferably by inclined segment wall surfaces of the wall regions, which taper upwards. Adhesion of the cells is also prevented by an upper side of the wall regions between the cell-receiving segments that tapers towards the wall tip.

Advantageously, the segment slit can fulfill several functions. Firstly, it additionally improves the partition between adjacent cell-receiving segments. By forming the segment slit with the lateral protrusions on the segment wall surfaces, the compartmentalization of the structured substrate surface can be improved. Cells are retained more reliably and for longer periods of time in the cell-receiving segments. Secondly, the segment slit allows optical observation of cells in the cell-receiving segment from the upper side of the cell substrate (or illumination for microscopic procedures), while the cells are simultaneously retained in the cell-receiving segment.

The segment slit can preferably have a smaller width than the width of the cell-receiving segment (groove) at its bottom, i.e. on the substrate body. Particularly preferably, the segment slit can have a width equal to or less than 20 μm, in particular equal to or less than 5 μm. With these dimensions, enclosing (trapping) of the cells, in particular of cell fibers, in the cell-receiving segments is advantageously achieved. The segment slit can be dimensioned in particular such that cells can be forced through the segment slit during their deposition on the cell substrate, for example under the effect of gravity or centrifugal forces, while they cannot pass through the segment slit again in the adherent condition in the cell-receiving segment without the effect of the forces occurring during deposition. This favors fixation of the cells in the cell-receiving segment. At the same time, cultivation medium can be supplied to the cells in the cell-receiving segment through the segment slit.

The lateral protrusions on the segment wall surfaces can preferably be dimensioned such that the segment slit has a width that is smaller than the diameter of the cells that are to be taken up by the cell substrate. For example, the segment slit to receive cells with a diameter of 10 μm can have a width <10 μm, for example 8 μm or less.

According to a further preferred embodiment of the invention, at least one, particularly preferably each, of the wall regions may comprise at least one through-opening between adjacent cell-receiving segments. The at least one through-opening forms an opening at a predetermined position along the longitudinal extension of a cell-receiving segment. The at least one through-opening is preferably oriented transversely to the longitudinal extension of the adjacent cell-receiving segments. Adjacent to the at least one through-opening, the associated cell-receiving segments are separated from one another along their longitudinal extension. The at least one through-opening can extend over the entire height of the wall region perpendicular to the substrate body or form an opening exclusively in the base area of the wall region. Advantageously, H-shaped connections can be created between cell-receiving segments, which provide lateral cross-linking of cell fibers (formation of tissue structures from individual fibers with lateral contacts). The at least one through-opening is preferably dimensioned such that connections of biological cells can form between the cell-receiving segments connected via the through-opening. The at least one through-opening can, for example, be dimensioned in with and height at least like a cross-sectional shape of the cell-receiving segments.

The wall regions can have through-openings at several predetermined positions, i.e. they can be interrupted, which allows targeted forwarding of action potentials to adjacent fiber structures. This can be advantageous for the simulation of excitation processes via two-dimensional tissue structures consisting of individual fibers contacting each other in these positions. A further advantage of the defined through-openings is the possibility of investigating complex excitation processes on these tissue structures using the coordinates of the transitions from action potentials to adjacent fibers. The through-openings in the wall regions, which otherwise separate individual fiber segments from each other, also allow axons and dendrites of nerve cells to grow through.

According to a further advantageous embodiment of the invention, at least one, particularly preferably each, of the wall regions can have a corrugated wall surface on at least one side. A lateral structure of the side surfaces of the wall regions can improve an adhesion of the cells in the cell-receiving segments and prolong the residence time of the cells.

Advantageously, the cell substrate offers the possibility of examining the cells taken up in the cell-receiving segments, whereby a plurality of different examination methods can be realized individually or in combination. Preferably, an optical examination, for example using a microscope, in particular a fluorescence measurement, an examination for recording the contraction or other movements of the cells, a chemical examination and/or an electrophysiological examination (recording electrophysiological features, in particular electrophysiological potentials, of the biological cells) can be provided.

If the cell substrate is configured for recording electrophysiological features of the biological cells, at least one of the cell-receiving segments can advantageously have an electrode arrangement with at least one electrode. Preferably, at least two electrodes can be provided, which are spaced apart from each other along the longitudinal extension of the cell-receiving segment. Advantageously, the at least one electrode can be arranged on the side of the cell-receiving segments facing the substrate body, in particular on their underside (bottom). Particularly preferably, more than two electrodes, for example three, four, five or more electrodes, can be arranged in each cell-receiving segment distributed along its longitudinal extension. Depending on the dimensions, even at least 10, in particular at least 100 or even more than 1000 electrodes can be arranged in each cell-receiving segment distributed along its longitudinal extension. Alternatively, a single electrode can be arranged in each cell-receiving segment. In this case, the cells are stimulated by applying a potential to an electrode in the cell-receiving segment, with the potential of a reference electrode (see e.g. FIG. 2) serving as the reference potential.

In addition to the aforementioned (measuring) electrodes, at least one reference electrode is provided on the cell substrate, which provides a reference potential, e.g. ground potential, for potential measurements on the cells.

According to an embodiment of the invention, a cell-receiving segment of a cell substrate can in particular have a first electrode at a first end of the cell-receiving segment, a second electrode between the first end and a second end of the cell-receiving segment, and a third electrode at the second end of the cell-receiving segment. The first electrode can be used to electrically stimulate biological cells in the cell-receiving segment, after which the speed of excitation conduction through the fiber is detected by recording field potentials with the second and third electrodes.

Advantageously, the electrode arrangement can be realized with different types of electrodes. In particular, the electrodes may comprise tip-shaped electrodes, which are configured to penetrate cell membranes and may also be referred to as protruding electrode tips, tip electrodes, nano- or microelectrodes, and/or flat surface electrodes, which are configured to derive field potentials at cell surfaces.

To provide the electrode arrangement of surface electrodes, an MEA substrate with an arrangement (preferably regular arrangement, e.g. matrix arrangement with straight rows and columns) of surface electrodes, each with an electrode line, can advantageously be used, wherein the structured substrate surface is formed on the surface of the MEA substrate. The structured substrate surface is shaped to match the arrangement of the surface electrodes so that the wall regions are formed between rows of electrodes and the surface electrodes of rows of electrodes are exposed at the bottoms of the compartments (cell-receiving segments). Advantageously, the surface electrodes of MEAs at the bottom of the compartments can be used to measure the longitudinal excitation propagation.

Advantageously, the electrode arrangement can comprise a CMOS-MEA. A CMOS-MEA comprises an MEA in which the electrodes are formed as CMOS semiconductors. CMOS-MEAs are manufactured, for example, by Multi Channel Systems MCS GmbH (Reutlingen, Germany). CMOS-MEAs can have 4000 or more electrodes on an area of one square millimeter, for example. A cell substrate with a CMOS-MEA can be manufactured, for example, by building up the wall regions on the surface of the CMOS-MEA, e.g. by printing them on.

According to a particularly advantageous embodiment of the invention, the electrode arrangement may be coupled to a measurement and evaluation device configured to characterize cell contacts between the cells depending on electrophysiological potentials detected with the electrodes. Advantageously, the measurement and evaluation device offers the possibility of deriving nexus contacts directly from measurement signals of the electrode arrangement of the cell substrate. Preferably, the measurement and evaluation device contains an amplifier for electrical signal amplification of the potentials discharged at the electrodes.

Advantageously, the allocation of one or more electrodes at the bottom of a cell-receiving segment enables a much more effective use of e.g. stem cell-derived nerve or muscle cells. For example, in a cell-receiving segment with a length of 1 mm and a width of 10 μm, only 5 to 100 cells with lengths of 200 μm (for spindle-shaped cells) or diameters of 10 μm (for round suspension cells) form a micromyocardial fiber whose individual cells are connected by nexus contacts. Independent measurements can be carried out on this fiber in the cell-receiving segment using optical methods (fluorescence probes) and/or electrophysiological measurements. With a seeding of approximately 50,000 cells on a conventional MEA, this means an approximately 1000-fold saving in cell material per measurement (per data point).

According to further preferred embodiments of the invention, at least one of the cell-receiving segments can have at least one bifurcation and/or a width varying along its longitudinal extension. Advantageously, further in vivo environmental conditions of cells or in particular cell tissue can be simulated with these variants, e.g. by bifurcations or step-shaped extensions or constrictions of the compartments. At a step-shaped extension or constriction, the width of the cell-receiving segment can change by a factor in the range of 2 to 10, for example. For example, anatomical structures of the myocardial tissue can be reproduced (modeled). Bifurcations of fibers can, for example, be the starting point of arrhythmias, so that a cell substrate with a bifurcated cell-receiving segment is particularly suitable for testing antiarrhythmic drugs (see e.g. [12]).

A bifurcation comprises an area of the structured substrate surface in which a segment portion of a cell-receiving segment splits into at least two segment portions and/or two segment portions merge into one segment portion. The bifurcation may comprise, for example, at least one of a Y-shaped bifurcation, a division forming right angles between the segment portions, and a division into curve-shaped segment portions. A bifurcation can also take place via a lateral through-opening of the wall region (H-shaped).

Optionally, at least one of the cell-receiving segments can be provided with an end wall at each of its longitudinal ends. Advantageously, this forms the cell-receiving segment as a compartment bounded on all sides along the lateral extension of the cell substrate. Each cell-receiving segment with end walls forms a defined vessel.

According to a further variant of the invention, at least two adjacent segment portions of at least one of the cell-receiving segments can be connected via a curve segment. This embodiment advantageously enables cell-receiving segments to be formed with a length that is greater than the extension of the cell substrate and/or with complex shapes. For example, a cell-receiving segment can have at least two straight segment portions which are connected via at least one bent curve portion, so that a cell-receiving segment can generally have a C-, S- or meander shape. The cell-receiving segments are arranged side by side in the substrate surface. The segment sections of a cell-receiving segment can preferably be arranged in such a way that they each run parallel to one another with their longitudinal extension (longitudinal direction).

The cell substrate according to the invention is configured to receive the biological cells in the cell-receiving segments. The biological cells may generally comprise plant or animal cells, preferably cells from mammals, in particular from humans, particularly preferably differentiated cells formed by differentiation of human pluripotent stem cells. The biological cells may have any shape and may, for example, be round or elongated. Connected cell groups (cell aggregates of two or more cells) can also be deposited on the cell substrate. In the suspended condition in the cultivation medium, the biological cells can have typical cross-sectional dimensions in the range of 5 μm to 80 μm, in particular 10 μm to 30 μm. On the cell substrate, the cells can be arranged singly or preferably as aggregates, in particular fibers, and/or, in particular when using bifurcated cell-receiving segments, form a tissue.

The cell substrate according to the invention is preferably also configured for cultivation (in particular growth and/or differentiation) of the biological cells in the cell-receiving segments. For this purpose, a supply of cultivation and/or differentiation media can be provided from the upper side of the cell substrate and/or through microfluidic lines in the substrate body. The provision of an enclosure of the area with the cell-receiving segments, e.g. by an annular wall, in particular made of glass or plastic, can be particularly advantageous for the supply of cultivation and/or differentiation media from the upper side of the cell substrate. The enclosure can, for example, have an internal diameter of 10 mm to 15 mm and a height of 3 mm to 15 mm and be set up for the supply of a cell suspension, a cultivation medium and/or test solutions from the upper side of the cell substrate.

To accommodate the cells, the cell-receiving segments can preferably have a width (distance between segment wall surfaces of adjacent wall regions facing the cell-receiving segment or optionally the width of the funnel slit) in the range equal to or greater than 2 μm, in particular equal to or greater than 5 μm, and/or equal to or less than 100 μm, in particular equal to or less than 30 μm. These ranges are advantageous, since for smaller widths there may not be sufficient space for cell growth and a supply of cultivation and/or differentiation medium, and for larger widths there may not be sufficient restriction to fiber growth. Particularly narrow cell-receiving segments are preferred to receive skeletal muscle fibers.

The cell-receiving segments can preferably have a depth perpendicular to the extension of the cell substrate (wall height of the wall region) in the range of equal to or greater than 5 μm, in particular equal to or greater than 30 μm, and/or equal to or less than 500 μm, in particular equal to or less than 300 μm. These ranges are advantageous, since for smaller depths there may not be sufficient space for cell growth and a supply of cultivation and/or differentiation medium and for greater depths observation of the cells, e.g. with a microscope, may be hindered.

A length of the cell-receiving segments can be selected in the range of equal to or greater than 300 μm, in particular equal to or greater than 500 μm, and/or equal to or less than 3000 μm, in particular equal to or less than 1000 μm. These ranges are advantageous, since for shorter lengths the fiber growth may be too limited and for longer lengths the fiber growth exceeds the lengths of cell fibers in a natural environment.

Preferably, the cell-receiving segments can have an aspect ratio which is formed by a quotient of a lateral width at the bottom of the cell-receiving segments and a depth of the cell-receiving segments and is selected in the range from 2 to 10.

Features disclosed in connection with the cell substrate or embodiments thereof are also preferred features of the methods according to the invention for using the cell substrate or embodiments thereof. The above-mentioned aspects and features according to the invention and preferred features, in particular with respect to the structure of the cell substrate and the dimensions and compositions of individual components described with respect to the cell substrate, also apply to the methods for its use. The preferred embodiments, variants and features of the invention described herein can be combined with one another.

### BRIEF DESCRIPTION OF THE DRAWINGS

Further details and advantages of the invention are described below with reference to the accompanying drawings, which show in schematic form:

FIG. 1: schematic sectional views of cell substrates (partial representations) with features of preferred embodiments of the invention;

FIGS. 2 and 3: schematic plan views of a MEA substrate with integrated surface electrodes;

FIG. 4: further sectional views of cell substrates (partial representations) with features of further embodiments of the invention;

FIG. 5: a plan view of a cell substrate with various forms of cell-receiving segments;

FIG. 6: a plan view of a cell substrate with an illustration of the detection of cell contacts by an electrophysiological measurement;

FIG. 7: a schematic sectional view of a cell-receiving segment with a surface electrode;

FIG. 8: a schematic sectional view of a cell-receiving segment with a tip electrode;

FIG. 9: a schematic illustration of the receiving of cells in a cell-receiving segment;

FIG. 10: a further illustration of the receiving of cells in cell-receiving segments; and

FIGS. 11 and 12: illustrations of conventional cell substrates (prior art).

### PREFERRED EMBODIMENTS OF THE INVENTION

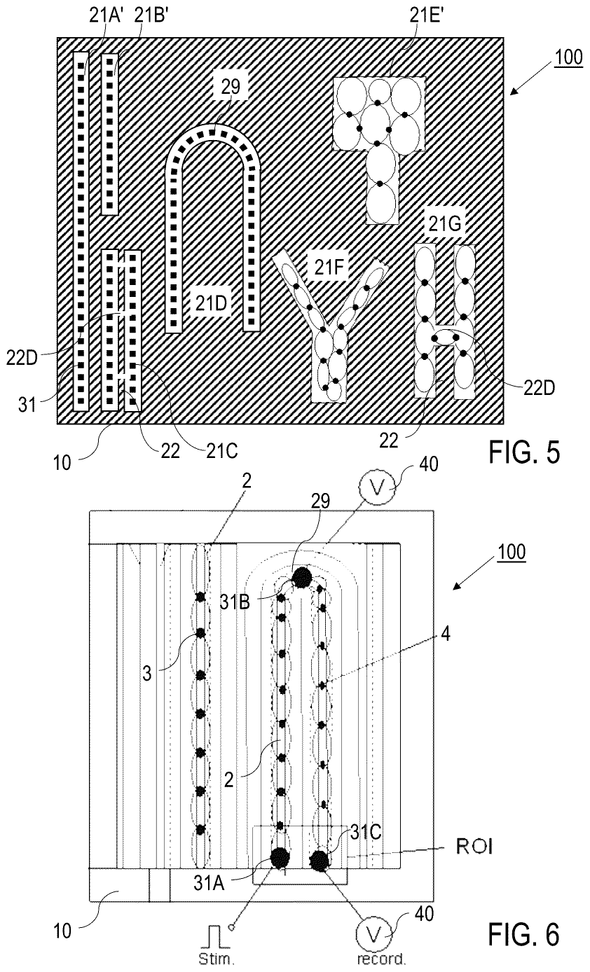

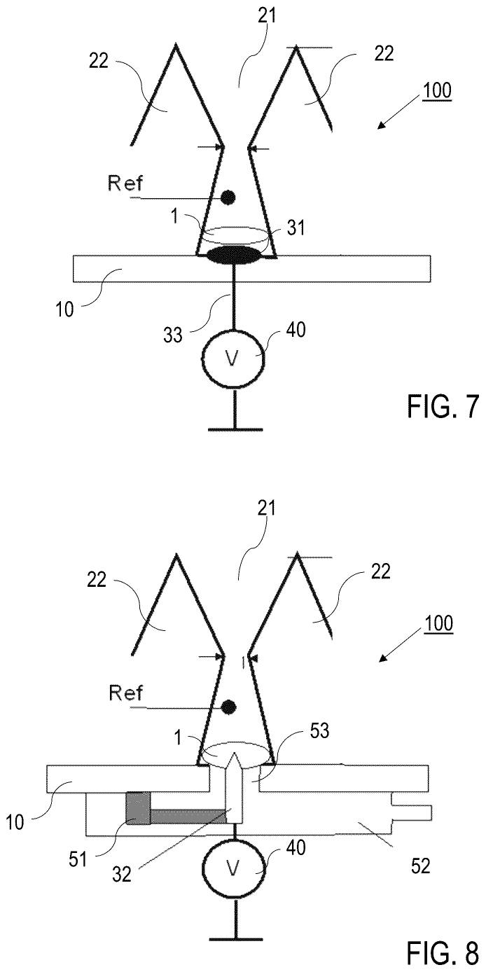

Features of preferred embodiments of the invention are described below, in particular with reference to the shape of cell-receiving segments (or segment sections thereof) and wall regions between these and the design of an electrode arrangement. Details of cell substrates or their use, such as details of the seeding of cells on the cell substrate, the cultivation or differentiation of cells, and/or the evaluation of electrophysiological measurement signals are not illustrated and described, insofar as they are known per se from the prior art. The invention is not limited to a particular embodiment of the cell substrate, but can be varied, e.g. with regard to the size and shape of the cell-receiving segments (or their segment sections) and wall regions between these or the number, arrangement and/or design of the electrodes or the number, arrangement and/or design of through-openings. The application of the invention is not limited to the measurements described with reference to FIGS. 6 to 8. The invention can also be used with other electrophysiological measurements or for other tasks without an electrophysiological measurement, such as spectroscopic investigations. For electrophysiological measurements, reference electrodes are provided, which are shown as examples in FIGS. 7 and 8.

The manufacturing of cell substrates according to the invention can be carried out using structuring techniques available per se for building up the structured substrate surface (bottom up, e.g. with laser-based rapid prototyping), and/or for forming the structured substrate surface by material removal (top down, e.g. with lithographic etching, e.g. UV lithography). Preferably, 2-photon polymerization (2-photon polymerization printing) with laser excitation can be used for rapid prototyping, especially for the production of the substrate surface (see [13] to [17]), which has advantages for the formation of the cell-receiving segments (or their segment sections) and wall regions with high accuracy and spatial resolution and optionally the formation of undercuts, e.g. when providing the funnel slits.

Surfaces of the cell substrate can be provided with coatings, such as Matrigel, fibronectin, laminin or collagen, in order to locally promote the adhesion and growth of cells.

The disadvantages of conventional cell substrates described above for the cultivation of myocardial or nerve fibers in particular are overcome by the wall regions shown in the figures, which are tip-shaped tapered at the top and/or formed by vertical ridges. When seeding a cell suspension, adhesion to the upper side of the wall regions is thus prevented. At the same time, sloping segment wall surfaces of the wall regions form an elongated funnel slit, which makes it easier to fill the cell-receiving segments, as the cells inevitably collect at the bottom of the cell-receiving segments due to gravity. This process can be supported by centrifugation.

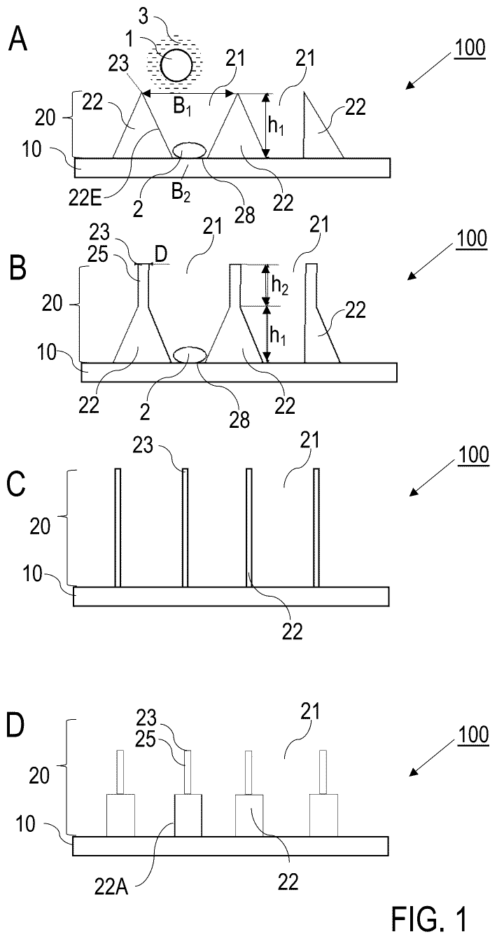

FIG. 1 schematically illustrates embodiments of cell substrates 100 (partial representations) in schematic sectional views, wherein a substrate body 10 and a structured substrate surface 20 are each shown with two or three cell-receiving segments 21, which are separated by wall regions 22 protruding from the substrate body 10. The sectional views show cross-sections of the cell-receiving segments 21 perpendicular to their longitudinal extension. The wall regions 22 between the adjacent cell-receiving segments 21 each have a triangular wall profile (see FIG. 1A), with which the cell-receiving segments 21 form a slot funnel shape on at least one of the lateral segment wall surfaces 22E bounding the wall regions 22, and/or the shape of a vertical ridge (see FIGS. 1B, 1C and 1D).

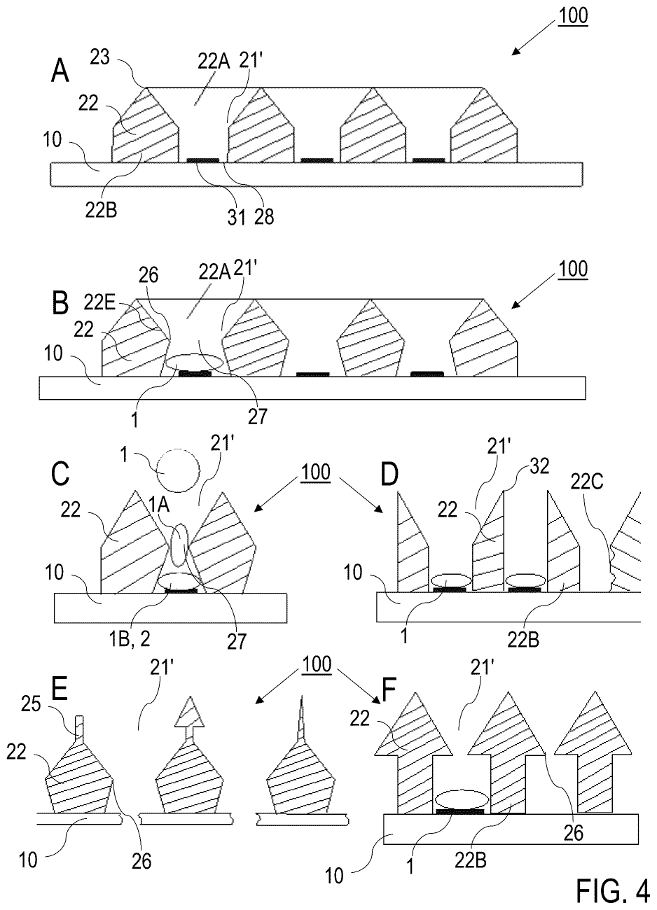

The wall profile of the wall regions 22 has a protruding wall tip 23, which is free-standing (FIG. 1A) or is formed by the vertical ridge 25 (FIGS. 1B, 1C and 1D). The vertical ridge 25 has the shape of a plane lamella or a more complex geometry with an arrow shape or a taper towards the free end of the vertical ridge 25 (see FIG. 4E).

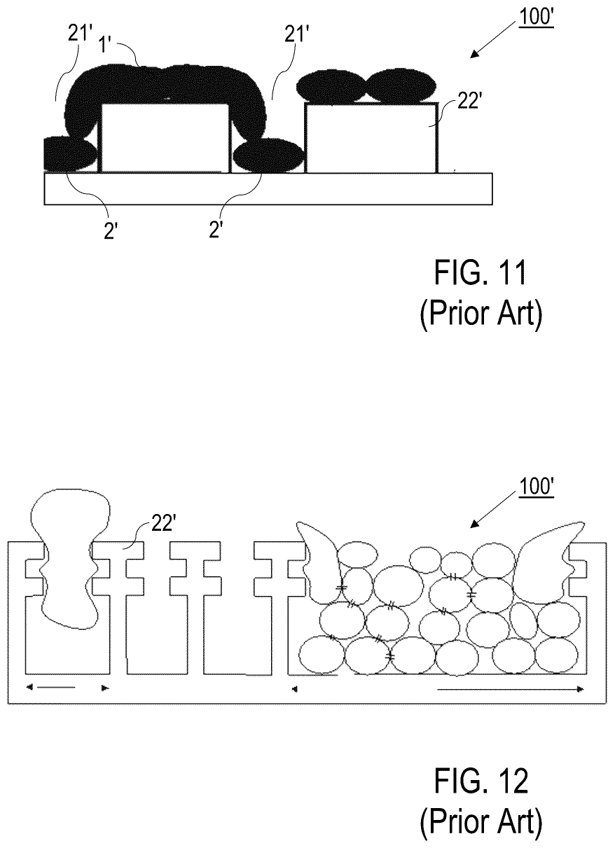

The comparison of the cell substrates 100 according to FIG. 1 with the conventional cell substrate 100′ according to FIG. 11 illustrates the effect of the cell substrate 100 according to the invention. In conventional cell substrates 100′, cells 1′ can adhere to flat upper sides of the partition walls 22′, which typically have a height of up to 50 μm, and overgrow the upper sides, so that contacts are formed between fibers 2′ in adjacent grooves 21′ (see FIG. 11) and independent examination of electrical processes on individual (adjacent) fibers becomes impossible. In contrast, the cell substrate 100 according to the invention suppresses a cell arrangement on the wall region 22.

According to FIG. 1A, the shape of the upwardly tapering wall regions 22 in the form of pointed roofs prevents cells 1 from adhering to the wall regions 22 during seeding of cells 1 from a suspension 3 (partially shown). The cells 1 sink under the effect of gravity to the bottom 28 of the cell-receiving segments 21, where they form fibers 2 as a result of further cultivation and/or differentiation. According to FIG. 1B, the vertical ridges 25 form the wall tips 23 and thus advantageously the desired barrier for cells 1 from adjacent cell-receiving segments 21, so that contacts between cell-receiving segments 21 are prevented. According to FIG. 1C, the vertical ridges 25 extend as ultra-thin walls over the entire height of the wall regions 20. According to FIG. 1D, the vertical ridges 25 are positioned as ultra-thin walls on wider, cuboidal base sections 22A.

In the variants shown in FIGS. 1 to 10, the substrate body 10 is formed, for example, from glass or a plastic, preferably with a thickness equal to or less than 1 mm. The bottom 28 of the cell-receiving segments 21 is formed by the substrate body 10, optionally with a coating to promote cell adhesion, e.g. with cell adhesion proteins, or an intermediate layer. The wall regions 22 are made, for example, from a polymer (see [13]-[17]) or, for example, from PDMS.

The dimensions of the structured substrate surface 20 of cell substrates 100 according to the invention can generally be selected, for example, in the following ranges: wall height h1 of the wall regions 22 (see e.g. FIG. 1A) ≥5 μm, in particular 10 μm to 100 μm, height h2 of the vertical ridge (see e.g. FIG. 1B) 10 μm to 100 μm, distance B1 of the wall tips 23 to 100 μm, width B2 of the bottom 28 3 μm to 50 μm, thickness D of the vertical ridges 25 0.5 μm to 10 μm, e.g. 1 μm to 5 μm or 5 μm to 10 μm, and width of the base section (see e.g. FIG. 1D) 10 μm to 20 μm.

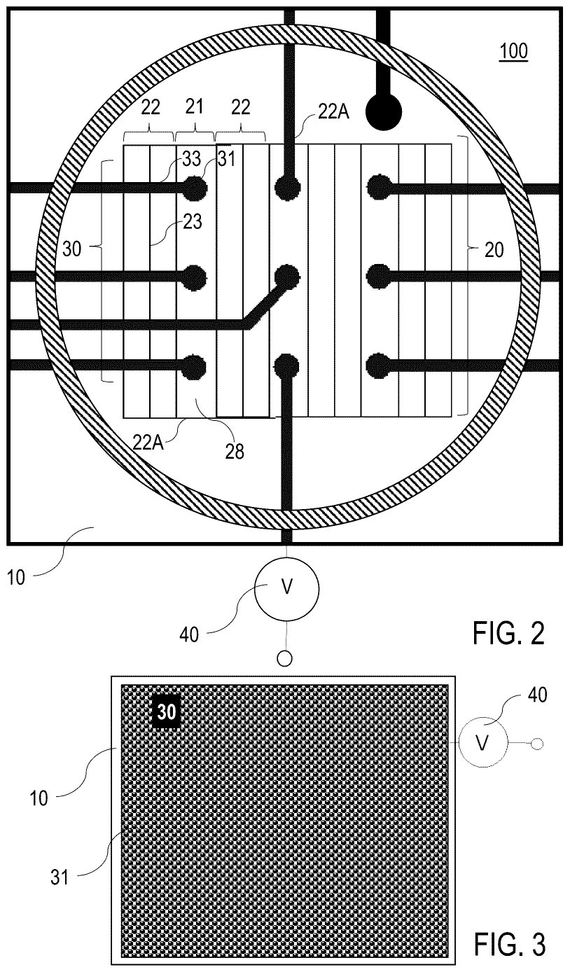

FIG. 2 schematically illustrates an embodiment of a cell substrate 100 in schematic plan view, wherein a substrate body 10 and a structured substrate surface 20 with three cell-receiving segments 21 are shown. The substrate body 10 is a MEA substrate (MEA chip) with an electrode arrangement 30, which has electrically separated surface electrodes 31, which are arranged at a distance from each other along the longitudinal extension of the cell-receiving segments 21, and a respective connection line 33 to each surface electrode 31. For reasons of clarity, the MEA substrate is shown with only 9 surface electrodes 31. In practice, a considerably higher number of e.g. 256 up to >1000 surface electrodes 31 can be provided.

Furthermore, a CMOS-MEA can be used as electrode device 30 on the substrate 10, which has, for example, more than 4000 deriving positions and is illustrated schematically in FIG. 3. In this embodiment, the substrate surface with the wall regions and cell-receiving segments can be formed directly on the electrodes 31 of the CMOS-MEA, for example by printing. The electrodes 31 of the CMOS-MEA can be coated, e.g. with a platinum layer (“black platinum”). According to FIG. 3, the electrode density can be much higher than in conventional MEAs (several thousand electrodes/mm²).

The wall regions and cell-receiving segments can preferably be printed directly on the electrodes 31 using 2-photon polymerization, unlike in FIG. 2. Each of the electrodes 31 is connected to a measurement and evaluation device 40.

The structured substrate surface 20 is formed with the wall regions 22 in such a way that the surface electrodes 31 are each exposed at the bottom of one of the cell-receiving segments 21. The wall regions 22 are formed, for example, with the exposed wall tips 23, which form continuous wall vertex lines over the length of the wall regions 22. At the longitudinal ends of the cell-receiving segments 21, end walls 22A are formed by the substrate body 10, with which the cell-receiving segments 21 are closed in the longitudinal direction (see also FIGS. 4A and 4B).

The surface electrodes 31 each comprise, for example, circular electrode layers which are made, for example, of metal, such as gold or titanium nitride, and have a diameter which is less than or equal to the width of the bottom 28 of the cell-receiving segments 21. The connection lines 33, via which the surface electrodes 31 are each connected to a separate measurement and evaluation device 40 (only one measurement and evaluation device 40 is shown, see also FIG. 7), comprise conductor tracks on and/or in the substrate body 10. The preferred material for the conductor tracks is, for example, transparent ITO (indium tin oxide), with SIN (silicon nitride), for example, serving as an insulating layer.

FIG. 2 illustrates an example of an annular enclosure 11 of the area with the cell-receiving segments 21, wall regions 22 and electrodes 31. The enclosure 11 has a height of e.g. 3 mm, so that a vessel is formed for holding cultivation media, cell suspensions and/or test substances limited to this area. The enclosure 11 can, for example, be made of a plastic and glued to the surface of the substrate body 10. Furthermore, the enclosure 11 can be connected to a fluidic device for supplying or removing liquids into or from the enclosure 11.

Further variants of the wall profile of the wall regions 22 according to embodiments of the invention are illustrated in FIG. 4. The wall profiles, which are shown as cross-sections, each delimit adjacent cell-receiving segments 21. Partial features of the individual variants can be combined, for example by providing the end walls 22A of FIGS. 4A and 4B and/or the corrugated wall surface 22C of FIG. 3D in one of the other variants.

According to FIG. 4A, the wall regions 22 each have a wall profile which comprises a rectangle at the base region 22B facing the substrate body 10 and a triangle with the roof tip 23 at the free end. Surface electrodes 31 are arranged on the bottoms 28 of the cell-receiving segments 21. The ends of the cell-receiving segments 21 are bounded by end walls 22A.

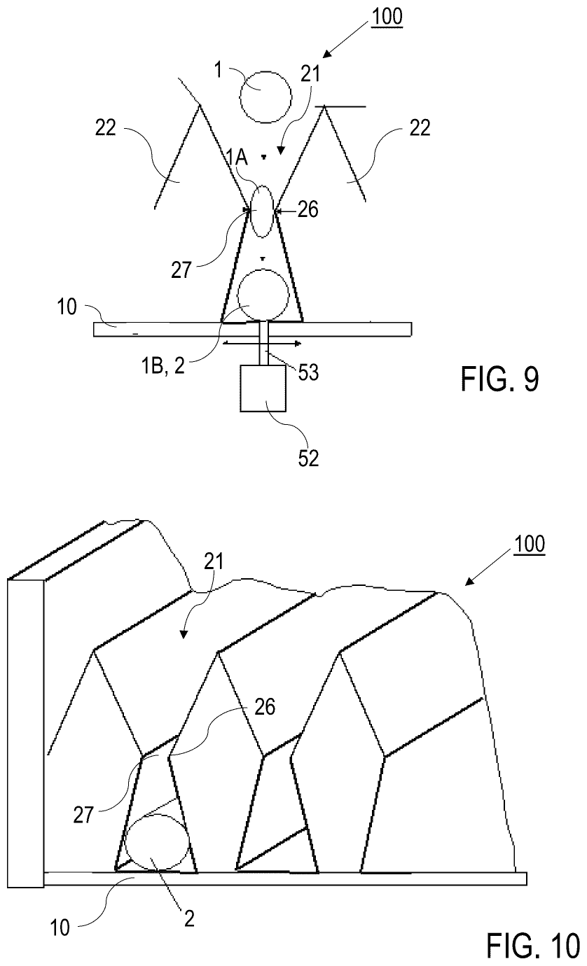

According to the variant shown in FIGS. 4B and 4C, the wall regions 22 each have a wall profile which comprises a tapering trapezoid in the base area 22B facing the substrate body 10 and the triangle with the roof tip 23 at the free end. With this geometry, lateral protrusions 26 are formed on the segment wall surfaces 22E and segment slits (funnel slits) 27 are formed between them. The segment slits 27 have a width that is smaller than the diameter of the cells 1. When passing through the segment slit 27, a temporary deformation of the cells 1 occurs (see 1A in FIGS. 4C and 9), which then expand again below the segment slit 27 and form a fiber 2 (see 1B, 2 in FIGS. 4C and 9) and are thus trapped in the cell-receiving segment 21 between the base regions 22B.

By varying the height of the base region 22B and the width of the separating slit 27, the geometry of the fibers to be formed can be determined. Typical dimensions are 10 μm to 30 μm for the width of the partition slit 27 and 1 μm to 50 μm for the height of the base region 22B, wherein these dimensions can vary depending on the fiber geometry specification.

FIG. 4D shows examples of further wall profiles of the wall regions 22, each with a triangular section, a wall tip 23 and a rectangular base region 22B, wherein at least one side wall in the base region 22B can have a corrugated wall surface 22C. With the corrugated wall surface 22B, the surface of the side wall is enlarged near the bottom 28, so that the fixation of the cells 1 and/or fibers in the cell-receiving segments 21 is additionally improved.

The vertical ridge 25 shown in FIG. 1B in the form of a plane lamella can be provided on a wall region 22 with a wall profile according to FIG. 4B, as shown in FIG. 4E (left). The profile of the vertical ridge 25 can have the shape of an arrow-shaped tip (FIG. 4E (center)) or a tapered tip (FIG. 4E (right)). In particular according to FIG. 4E (right), the radius of curvature of the vertical ridge 25 can advantageously be reduced to a radius of curvature of 0.5 μm.

FIG. 4F shows a further variant of the wall regions 22 with protrusions 26, which are formed by a rectangular base region 22B and a triangular tip. Since the wall region 22 has a smaller thickness at the base region 22B than at the protrusion 26 and an undercut is formed, a larger space to receive cells can be provided at the bottom of the cell-receiving segment 21.

The variants with narrowing segment slit 27 (FIG. 4B, C, E and F) are preferably used if the cells 1 are to be deposited in the cell-receiving segments 21 by means of centrifugation. Since greater forces occur during centrifugation than during sedimentation with the sole effect of gravity, the deformation of the cells 1 is supported as they pass through the segment slit 27.

The plan view of a cell substrate in FIG. 5 schematically illustrates variants of cell-receiving segments according to embodiments of the invention. Cell-receiving segments can extend over the entire extension of the cell substrate 10 (cell-receiving segment 21A), be formed shorter than the extension of the cell substrate (cell-receiving segments 21B, 21C), form a C-shape with two straight segment portions and a curve portion 29 (curve-shaped cell-receiving segment 21D), have a step-shaped extension (cell-receiving segment 21E), form at least one Y-shaped bifurcation (cell-receiving segment 21F) and/or form at least one H-shaped bifurcation (cell-receiving segments 21C, 21G).

Cell-receiving segments 21C, 21G with at least one H-shaped bifurcation are formed by the wall region 22 between adjacent cell-receiving segments having at least one through-opening 22D. A wall interruption is formed by the through-opening 22D, so that the adjacent cell-receiving segments are H-shaped in plan view. Several through-openings 22D can be used to connect the adjacent cell-receiving segments, e.g. of a MEA (FIGS. 2, 3), at defined positions, which is particularly advantageous for the simulation of myocardial tissue structures. For example, there are also lateral connections in heart muscle tissue, where cell fibers can also be connected laterally via nexus contacts.

The variants are shown in FIG. 5 as single or paired cell-receiving segments. Combinations, sub-combinations or variations of these variants can be formed. In practice, a plurality of cell-receiving segments is preferably provided, wherein all cell-receiving segments represent the same variant or cell-receiving segments of different variants are combined. The cell-receiving segments are formed on the substrate body 10 and arranged side by side, so that adjacent cell-receiving segments are each separated by a wall region 22 protruding from the substrate body 10. For reasons of clarity, the cell-receiving segments are shown in FIG. 5 with relatively large distances between them and without the structure of the wall regions. In practice, they can alternatively be arranged with smaller distances. In any case, the material between the cell-receiving segments is formed in the shape of the wall regions 22 shown, for example, in FIGS. 1 to 4.

The cell-receiving segments 21A to 21C exemplarily show the row-shaped arrangement of a plurality of electrodes, e.g. surface electrodes 31. The cell-receiving segment 21D exemplarily illustrates that the cell-receiving segments are generally not necessarily single cells or cell fibers, but alternatively may be configured to receive planar cell groups (cell layers) and/or volume-shaped cell groups (cell aggregates).

FIG. 6 shows a schematic plan view of a further exemplary embodiment of the cell substrate 100 with a straight cell-receiving segment 21A and a curve-shaped cell-receiving segment 21C, which are each bounded by wall regions, e.g. in the form of the wall regions 22 shown in FIGS. 1 to 4, preferably by wall regions 22 with protrusions 26 as shown in FIG. 4B, 4C, 4E or 4F. Fibers 2 from cardiac cells are preferably held in the cell-receiving segments 21A, 21C below the protrusions 26 on both sides of the adjacent wall regions, which makes it more difficult for the fibers 2 to detach from the cell substrate 100.

The straight cell-receiving segment 21A in the left part of FIG. 6 contains a fiber 2 of cardiac cells whose nexus contacts 4 are schematically illustrated. Measurements of field potentials and stimulations of the fibers can take place at different positions along the longitudinal extension of the fibers. The length of fiber 2 is 3 mm, for example. With the curve-shaped cell-receiving segment 21C in the right part of FIG. 6, the length of the fiber 2 can be extended to 7 mm, for example.

The curve-shaped cell-receiving segment 21C in the right part of FIG. 6 has a first surface electrode 31A at a first end of the cell-receiving segment 21C, a second surface electrode 31B in the curve portion 29 and a third surface electrode 31C at the second end of the cell-receiving segment 21C. With the first surface electrode 31A, a pulse-shaped electrical stimulation of the biological cells in the cell-receiving segment 21C can take place at a starting time by means of a pulse voltage source (not shown). By detecting field potentials with the second and third surface electrodes 31B, 31C, which are each connected to the measurement and evaluation device 40, the run times of the excitation relative to the start time and thus the speed of the excitation conduction through the fiber are detected.

The ends of the cell-receiving segment 21C can alternatively or additionally be detected with an optical measuring device, in particular a microscope. The observation field ROI (region of interest) of the optical measuring device (not shown) contains the ends of the cell-receiving segment 21C. A stimulation pulse and the arrival of the excitation at the second end of the cell-receiving segment 21C can be detected, for example, by measuring fluorescence signals (e.g. with a “FluoVolt™” probe) in a camera segment (ROI, region of interest).

FIGS. 7 and 8 schematically illustrate the arrangement of electrodes in a cell-receiving segment 21. FIGS. 7 and 8 show a partial sectional view of a cell substrate 100 with two wall regions 22, which are formed according to FIG. 4B, and the cell-receiving segment 21 between the wall regions 22.

According to FIG. 7, a surface electrode 31 for measurements of field potentials (surface field potentials), as is known from conventional measurements with MEAs, is located at the bottom 28. The surface electrode 31 is connected to a measurement and evaluation device 40, e.g. a voltage measuring device, via a connection line 33 embedded in the substrate body 10. Potential measurements on the cell 1 are carried out with respect to a reference electrode (Ref), which is in electrically conductive contact with the cultivation medium in the interior of the cell-receiving segment 21.

FIG. 8 schematically illustrates the measurement of intracellular potentials with an electrode tip 32, as described in [6], for example. The electrode tip 32 penetrates a cell membrane of a cell 1 in the cell-receiving segment 21. The electrode tip 32 is mechanically moved into a perfusion pore 53 in the substrate body 10 with the aid of a coupled manipulator 51, e.g. a piezoelectric actuator, of a suction device 52, which leads to penetration of the membrane of the cell 1 with the electrode tip 32. Alternatively, the membrane can be penetrated by electroporation. Potential measurements on the cell 1 are again carried out with respect to a reference electrode (Ref), which is in electrically conductive contact with the cultivation medium in the interior of the cell-receiving segment 21.

The suction device 52 and the perfusion pore 53 in the substrate body 10 can also be provided without the provision of an electrode tip 32, as illustrated schematically in FIG. 9. The suction device 52 can also be used to additionally hold the cell 1B or fiber 2 in the cell-receiving segment 21 and/or to accelerate the filling of the cell-receiving segment 21.

The embodiment of the cell substrate shown in FIG. 8 can be used as a generator device (“biological battery”) for generating electrical current by deriving membrane potential (potentials in the range of the cell membrane potential of e.g. 40 mV to 100 mV) on the biological cells 1 in the cell-receiving segments 21 of the cell substrate 100. The generator device is configured with an MEA, for example, like the embodiment according to FIG. 2 or 3, wherein for this application a larger number of cells adapted to the function of the generator device is integrated on a correspondingly larger surface and supplied with culture media. Myocardial cells are particularly suitable for use as generator device due to their efficient metabolism.

Furthermore, FIGS. 9 and 10 illustrate the effect of the protrusions 26 to form a narrowing segment slit 27 during the deposition of cells 1 in the cell-receiving segment 21. The initially spherical cell 1 in suspension (FIG. 9) is forced through the segment slit 27 under the effect of a centrifugation force, for example, whereby it is deformed without being damaged (1A). In the area of the undercut below the protrusion 26, the cell can then relax again and assume the round shape (1B) or form a fiber (2), a cell layer or a cell aggregate. The fiber 2, which extends in the longitudinal direction of a cell-receiving segment 21, is also shown schematically in FIG. 10.

The features of the invention disclosed in the above description, the drawings and the claims may individually, in combination or sub-combination be of importance for practicing the invention in its various embodiments.

1. A cell substrate configured to receive biological cells, comprising
a substrate body having a structured substrate surface, wherein
the structured substrate surface has a plurality of cell-receiving segments arranged side by side, each having a longitudinal extension,
adjacent cell-receiving segments are each separated by a wall region projecting from the substrate body and bounded by segment wall surfaces, wall regions between the adjacent cell-receiving segments each have a wall profile perpendicular to the longitudinal extension of the adjacent cell-receiving segments, and
the wall profile of the wall regions between the adjacent cell-receiving segments in each case has a wall tip which projects from the substrate body and suppresses a cell arrangement on the wall region.

2. The cell substrate according to claim 1, wherein
distally at the wall tip between adjacent cell-receiving segments, the segment wall surfaces of at least one of the wall regions enclose an acute angle or run parallel.

3. The cell substrate according to claim 2, wherein
the segment wall surfaces enclose the acute angle and extend obliquely relative to each other and/or relative to an extension of the substrate body.

4. The cell substrate according to claim 1, wherein
the segment wall surfaces have a low roughness such that structures on the segment wall surfaces have dimensions<100 nm,

5. The cell substrate according to claim 1, wherein
at least one of the wall regions has a vertical ridge projecting from the substrate body, wherein the vertical ridge forms the wall tip in the wall profile of the wall region.

6. The cell substrate according to claim 5, wherein
the vertical ridge extends over an entire height of the wall region.

7. The cell substrate according to claim 1, wherein
the segment wall surfaces adjacent to at least one of the cell-receiving segments have lateral protrusions facing each other and forming a segment slit extending along the longitudinal extension of the cell-receiving segments.

8. The cell substrate according to claim 7, wherein
the segment slit has a width equal to or less than 10 μm.

9. The cell substrate according to claim 1, wherein
the segment wall surfaces adjacent to at least one of the cell-receiving segments form an elongate funnel slit.

10. The cell substrate according to claim 1, wherein
at least one of the wall regions has at least one through-opening between the adjacent cell-receiving segments.

11. The cell substrate according to claim 1, wherein
at least one of the wall regions has a corrugated wall surface on at least one side.

12. The cell substrate according to claim 1, which is configured to detect electrophysiological features of the biological cells, wherein
at least one of the cell-receiving segments has an electrode arrangement with at least one electrode.

13. The cell substrate according to claim 12, wherein
the electrode arrangement has at least two electrodes which are arranged spaced apart from one another along the longitudinal extension of the at least one of the cell-receiving segments.

14. The cell substrate according to claim 12, wherein
the at least one electrode is arranged at a bottom of the at least one of the cell-receiving segments.

15. The cell substrate according to claim 12, wherein
the at least one electrode comprises at least one of surface electrodes configured for a planar contact with the biological cells and protruding electrode tips configured for intrusion into the biological cells.

16. The cell substrate according to claim 12, wherein
the electrode arrangement comprises a CMOS-MEA.

17. The cell substrate according to claim 12, wherein
the electrode arrangement is coupled to a measurement and evaluation device configured to characterize cell contacts between the biological cells depending on electrophysiological potentials detected with the at least one electrode.

18. The cell substrate according to claim 1, having at least one of the features
at least one of the cell-receiving segments has at least one bifurcation
at least one of the cell-receiving segments is provided with an end wall at each of its longitudinal ends, and
at least one of the cell-receiving segments has a varying width along its longitudinal extension.

19. The cell substrate according to claim 1, wherein
at least two adjacent segment portions of at least one of the cell-receiving segments are connected via a curve portion.

20. The cell substrate according to claim 1, wherein
the cell-receiving segments have an aspect ratio which is formed by a quotient of a lateral width at a bottom of the cell-receiving segments and a depth of the cell-receiving segments and is selected in a range from 2 to 10.

21. A method of using the cell substrate according to claim 1, comprising at least one of the steps of
cultivation of the biological cells in the cell-receiving segments of the cell substrate,
examination of the biological cells,
measurement of electrophysiological potentials on biological cells in the cell-receiving segments of the cell substrate,
determination of an excitation propagation speed through the biological cells along at least one of the cell-receiving segments of the cell substrate by a detection of a time-delayed arrival of an electrical excitation at electrodes at a bottom of the cell-receiving segment,
determination of an excitation propagation speed through the biological cells along at least one of the cell-receiving segments of the cell substrate,
determination of an excitation propagation speed through the biological cells along at least one of the cell-receiving segments of the cell substrate with simultaneous fluorescence measurement for forwarding an action potential with a potential-sensitive fluorescence probe and an intracellular calcium release with a calcium-sensitive fluorescence probe, a cell contraction with video microscopy,
optical measurement of a contraction of heart muscle cells by analyzing athe shortening of cell segments,
measurements of intracellular calcium concentrations,
electrical stimulation of the biological cells in the cell-receiving segments of the cell substrate,
biochemical stimulation of the biological cells in the cell-receiving segments of the cell substrate, and
operation of the cell substrate as a generator device configured to generate electric current by membrane potential discharge at the biological cells in the cell-receiving segments of the cell substrate.
